(19) United States
(12) Patent Application Publication
(10) Pub. No.: US 2001/0049547 A1
(43) Pub. Date: Dec. 6, 2001

(54) Stent introducer apparatus

(76) Inventor: Scott T. Moore, Rural Hall, NC (US)

Correspondence Address: Charles W. Agnew, Patent Agent, P.O. Box 2269, Bloomington, IN, 47402-2269, US

(21) Appl. No.: 09/777,223

(22) Filed: Feb. 5, 2001

Related U.S. Application Data
(60) Provisional application No. 60/180,453, filed on Feb. 4, 2000.

(51) Int. Cl. A61F 2/06

(52) U.S. Cl. 623/1.11

(57) ABSTRACT

Disclosed is a stent introducer apparatus comprising a introducer catheter, usually comprising clear polytetrafluoroethylene, and a pusher assembly that is configured to be able to deliver a stent, such as a self-expanding stent, within a tortuous duct or vessel, even if the introducer catheter becomes kinked during the procedure. In an embodiment for use in the biliary system, the pusher assembly includes a first tubular portion, comprising a material with high column strength, such as polyetheretherketone, and a shorter second tubular portion, which is made of a highly flexible material such as metal-braided polyimide or nititnol tubing, that is divided into a distal, stent-carrying section and a proximal, flexible section. The second tubular portion may be made of a smaller diameter that the first tubular portion to reduce possible impingement by the introducer catheter is the latter kinks during a procedure. At the junction between the stent-carrying and flexible sections is a pusher member to urge the stent from the distal end of the introducer catheter. In one aspect of the invention, the distal tip and pusher member tightly hold the stent to eliminate gaps so that the likelihood of the introducer catheter kinking at the contact point between the pusher member and stent is greatly reduced.

### CROSS-REFERENCE TO RELATED APPLICATIONS

This application claims priority of provisional application Serial No. 60/180,453, filed Feb. 4, 2000.

### TECHNICAL FIELD

This invention relates to medical devices, more particularly to an apparatus for delivering an implantable prosthesis.

### BACKGROUND OF THE INVENTION

Placement of a stent within the biliary tree can be problematic in that the catheter delivery system must make a severe turn from the duodenum into the ostium in order to access the common bile duct. Current biliary and pancreatic stent delivery systems comprise an introducer catheter with the stent loaded at the distal end. A pusher catheter is used to deploy the stent from introducer catheter. Physicians strongly prefer that the delivery catheter be made of a clear material in order that they can see the stent within the catheter. This usually requires that the catheter be made of polytetrafluoroethylene (PTFE) which by the nature of the material, makes the catheter predisposed to kinking. When the introducer catheter kinks, it can impinge on the pusher catheter, preventing it from being able to advance the stent from the outer catheter. While the stent and pusher catheter serve to fill the lumen of the introducer catheter, making kinking within these portions less of a problem, the junction between stent and pusher is vulnerable point on the catheter where a severe kink can occur. If so, the pusher may not be able to traverse the catheter stricture to advance the stent. Some manufacturers avoid this problem because they use an axially contracting stent which overlaps with the distal end of the pusher, resulting in the most likely kinking point being reinforced by the stent and pusher from within. However, this system has other disadvantages in that stents that shorten are less desirable than non-contracting stents because of difficulty in placement. Non-shortening biliary stents, such as the ZA-STENT™ or SPIRAL Z™ Biliary Stents (Wilson-Cook Medical, Inc., Winston-Salem, N.C.), can be placed more accurately and provide superior coverage; however, the point on the catheter most susceptible to kinking is not protected by the stent, making kinking more of serious concern when PTFE is used for the introducer catheter. Another common problem with current biliary stent delivery systems is diminished recapture capability—the inability to retrieve the introducer system following stent delivery without having it become entangled within the stent or upon the introducer catheter itself. What is needed is a biliary and pancreatic stent introducer system that can still be deployed when the outer catheter kinks and that can be easily removed once the stent is deployed.

### SUMMARY OF THE INVENTION

The foregoing problems are solved and a technical advance is achieved in a stent introducer apparatus having a two-part pusher assembly with a lumen therethrough for introduction of a wire guide. The pusher assembly can be used to deploy a preloaded self-expanding stent from the distal end of an introducer catheter, such as a PTFE introducer sheath used to delivery a biliary or pancreatic stent. The pusher assembly comprises a first or proximal tubular portion that substantially fills the introducer catheter lumen and is made of a material with superior column strength, such as polyetheretherketone (PEEK), and a second or distal tubular portion which has a combination of good column strength and superior flexural properties, such as braided polyimide or nitinol, to distribute the severe bending force more evenly along the introducer catheter and help reduce the severity of kinking. Located at a point along the second tubular portion of the pusher assembly is a pusher member designed to urge the stent forward. The pusher member can comprise one or more separate elements attached to the second tubular portion or it can be an integral modification thereof than provides a mechanism for advancing or deploying the stent. In one embodiment, the pusher member comprises a pusher head made of metal or an insert-molded polymer that provides a broad surface for applying force to advance the stent. Typically, the stent is loaded while applying pressure against the pusher head to reduce any gap therebetween and help force any kinks experienced during the procedure to occur proximal to the pusher member, thereby not interfering with the ability of the pusher assembly to advance the stent from the introducer catheter.

In another aspect of the invention, the pusher member is configured such that the proximal portion of the pusher member can more easily negotiate a kink in the introducer catheter during withdrawal of the pusher assembly following delivery. This can be accomplished by tapering the distal tubular portion. In the illustrative embodiment, a similar proximal taper occurs on the distal tip of the pusher assembly, located distal to the stent. The face of the pusher member contains a chamfer to help prevent it from digging into the inner wall of the introducer catheter. In one embodiment, there is a second member at the junction between the second tubular portion and the first tubular portion. This second member is tapered distally to help facilitate its advancement through any kink that might occur along the section of the introducer catheter that is distal to that point.

### BRIEF DESCRIPTION OF THE DRAWINGS

FIG. 1 depicts a partially sectioned side view of an illustrative embodiment of the present invention;

FIG. 2 depicts a enlarged cross-sectional view of the embodiment of FIG. 1;

FIG. 3 depicts a partially sectioned view of the embodiment of FIG. 1 in a kinked introducer catheter;

FIG. 4 depicts a partially sectioned view of a second embodiment of a pusher member of the present invention;

FIG. 5 depicts a cross-sectional view of an embodiment of the present invention in which the second tubular portion extends at least substantially the length of the first tubular portion; and

FIGS. 6-7 depict cross-sectional views two embodiment of the present invention in which the first and second tubular portions or the pusher assembly comprise a single member.

### DETAILED DESCRIPTION

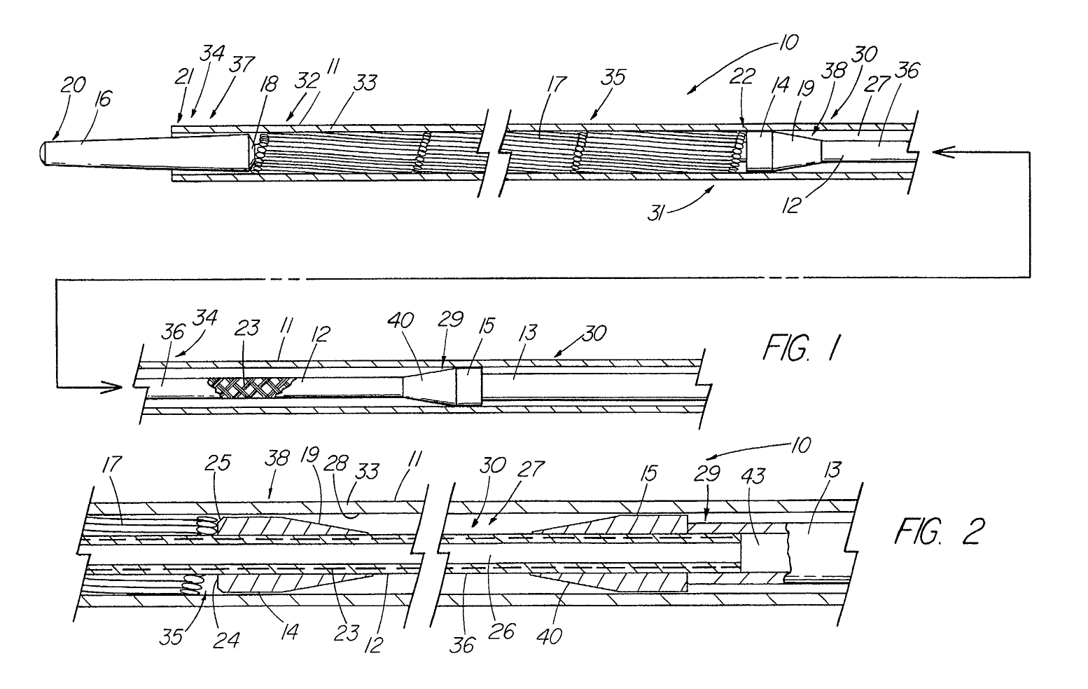

The present invention comprises a stent introducer apparatus 10, an illustrative embodiment of which is depicted in FIGS. 1-2. The stent introducer apparatus 10 comprises a pusher assembly 30 for advancing a stent 17 for deployment within a duct or vessel. In embodiment depicted in FIG. 1, the stent is a self-expanding biliary stent such as the COOK SPIRAL Z™ Stent; however, the type of stent is not considered important to the understanding of the invention. In the example in FIG. 1, the minimum size of the introducer catheter typically ranges from 8.0 to 8.5 FR (2.67 to 2.83 mm), depending on the stent used. The SPIRAL Z™ Biliary Stent, being somewhat larger than the ZA-STENT™ Biliary Stent, requires the larger introducer, while the smaller stent can be deployed from either sized introducer.

As depicted in FIGS. 1-2, the stent introducer apparatus 10 may further include an introducer catheter 11, which in the illustrative embodiment, is made primarily of a substantially clear polymer such as PTFE. The pusher assembly 30 and the preloaded stent 17 are coaxially disposed within passageway 27 of the introducer catheter 11 with the stent 17 residing in the distal portion 34 of the introducer catheter until it is expelled from the distal end 21 thereof by advancement of the pusher assembly 30 or withdrawal of the introducer catheter 11.

The pusher assembly of FIGS. 1-2 comprises a first or proximal tubular portion 13 and a second or distal tubular portion 12. The first and second tubular portions 12,13 can be formed as separate members and attached, or represent different portions of a single member, each having different physical properties. Each portion 12,13 has a lumen extending therethrough that is sufficiently large for accommodating an ancillary device such as a 0.035″ (0.89 mm) wire guide. The first tubular portion 13 can comprise a rigid or non-rigid member or portion thereof, depending on the application. In the illustrative embodiment, the first tubular portion 13 comprises a non-rigid polymer tube made of a material with superior column strength. Possible materials include, but are not limited to PEEK, polyvinyl chloride (PVC), polyimide, and polyurethane. The O.D. of the first tubular portion 13, approximately 0.07″ (1.78 mm) in the illustrative example, is such that it takes up most of the I.D. of the passageway 27 of the introducer catheter 11, thereby providing support thereto and reducing the likelihood and severity of kinking in the introducer catheter 11. Maximizing the pusher catheter O.D. also adds column strength for pushing the stent from the catheter. The second tubular portion 12 extends distally from the first tubular portion 13, to which it is joined, and comprises a tube made of a flexible material, also with sufficient column strength to allow the pusher assembly 30 to advance the stent from the introducer catheter 11. In the illustrative embodiment, the second tubular portion 12 comprises a polyimide tube reinforced with a stainless steel braid. Other possible materials include PEEK or metal tubing such as nitinol or stainless steel, depending on the degree of bending that the introducer must undergo. Nitinol tubing exhibits good laterally flexibility and kink-resistance, but is generally stiffer than braided polyimide tubing. Both the pusher assembly 30 and the introducer catheter 11 are connected at their proximal ends to a well-known coaxial medical device handle (not illustrated) that permits the pusher assembly 30 to be advanced relative to the introducer catheter 11 for deployment of the stent 17. An example of a suitable slider-type handle can be found on the previous-generation delivery systems for the Wilson-Cook SPIRAL Z™ and ZA-STENT™ Biliary Stents.

As a means to push the stent 17 out of the introducer catheter, a pusher member 14 is affixed to, integrally formed with the second tubular portion 12. In the illustrative embodiment, the pusher member 14 comprises a pusher head that includes a broad face 24 to contact the proximal end 31 of the stent and urge the stent forward until deployment has been achieved. The illustrative pusher member 14 can be made of metal such as 303 or 304 stainless steel, or it can comprise a polymer that is insert molded, bonded, or otherwise attached to the second tubular portion. The O.D. of the pusher member generally depends on the type of stent to be delivered. In the illustrative example, a SPIRAL Z™ Biliary Stent, which is deliverable through a 8.5 Fr (2.83 mm) introducer catheter, would have a 0.088″ (2.24 mm) O.D. pusher member 14. The ZA-STENT™ Biliary Stent, which can be introduced through either a 8.0 or 8.5 Fr (2.67 or 2.83 mm) introducer, could have a 0.077″ O.D. (1.96 mm) pusher member 14 if the 8.0 Fr (2.67 mm) introducer is used. The dimensions of the pusher member 14 could vary further, depending on a number of factors, particularly the I.D. of the introducer catheter lumen 27. Because of the desirability of having the pusher member 14 diameter be as close to the I.D. of the introducer catheter lumen 27 as possible, an optional chamfer 25 is included at the outside edge of the face 24 to help prevent the pusher member 14 from digging into the inner wall 28 of the introducer catheter 11 during advancement. In the illustrative embodiment, the pusher member 14 is placed over and glued to the second tubular portion 12 such that the contact point 22 between the two lies at an intermediate point along the second tubular portion 12. In the illustrative embodiment, the pusher member 14 represents a junction 38 between two sections of the second tubular portion 12. Proximal to the pusher member 14, lies the flexible section 36 of the second tubular portion 12, while distal to the contact point 22 lies the stent loading section of the second tubular portion 12. While these two sections 35,36 comprise a single piece of reinforced polyimide tubing in the illustrative embodiment, it is also possible that they be constructed with different materials or properties insomuch that each section 35,36 is likely to experience bend stresses during introduction due to the presence of the preloaded stent 17 over the stent loading section 35. The length of the stent loading section 35 corresponds to the length of the stent 17. A distal tip 16, made of PEBAX® (Atofina Chemicals, Philadelphia, Pa.) or a similar soft polymer with good bonding properties, is bonded to the distal end 37 of the second tubular portion 12 after the stent 17 has been preloaded thereon. The distal tip 16 may include barium sulfate or some other agent or marker to provide radiopacity. Both the distal tip 16 and distal end 21 of the catheter are rounded for atraumatic entry into the bile duct.

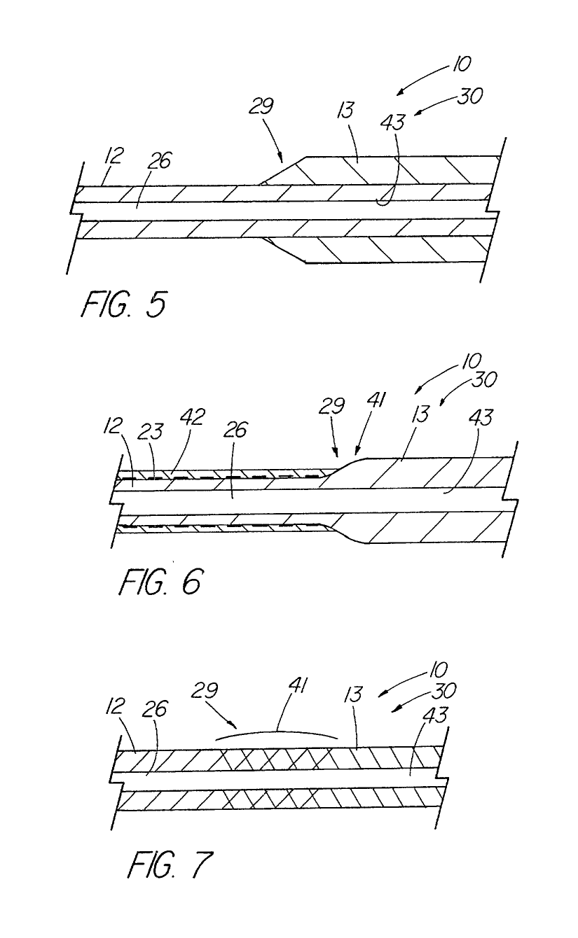

The two-part pusher assembly 30 provides an advantageous combination of both strength and flexibility that is desirable for biliary access. The section of the second tubular portion 12 proximal to the contact point 22 provides the stent introducer apparatus 10 with the ability to make a tortuous bend, such as into the ostium of the common bile duct, by distributing the bending stresses over a large area (approximately 20 cm in the illustrative embodiment). In the illustrative embodiment, the second tubular portion 12 is made to have a smaller O.D., approximately 0.045″ (1.14 mm), to increase laterally flexibility. The first tubular portion 13 comprises the majority of the pusher assembly 30 because of the increased column strength and protection to the introducer catheter 11 it provides. For example, a pusher assembly 30 might measure 190 cm from the proximal end of the catheter (distal end of the handle) to the proximal end 31 of the stent 17, wherein 160 cm of this length might comprise the first tubular portion 12 with only 30 cm comprising the flexible section 36 of the second tubular portion 12. Generally, the flexible section should comprise about 10-20% of the pusher assembly 30 in biliary applications. For other applications, the actual length of the flexible section can be vary, depending on the application. For example, the entire stent introducer apparatus 10 could be made smaller for deploying vascular stents, or it could have utility in placing colonic stents where the anatomy can also produce severe angle that can be of concern. For biliary applications, the distance from the junction between the handle and catheter to the distal end 20 of the introducer apparatus should generally measure at least 200 cm for a typical adult patient. As shown in FIG. 2, the second tubular portion 12 is attached to the first tubular portion 13, by a well-known bonding method, such as gluing. In the illustrative embodiment, a second member 15, such as a band similar to pusher member 14, and which is made of metal or plastic, is placed at the junction 29 between the distal and first tubular portions 12,13 and glued in place with the two portions overlapping each other by approximately 3-5 mm. FIG. 5 depicts an embodiment in which the second tubular portion 12 extends the entire length (or nearly the entire length) of the first tubular portion 13 such that the latter portion is essentially providing column strength and kink resistance (especially because of the increased diameter) to the proximal or remaining portion of the pusher assembly 12 proximal to initial junction 29 point. The second tubular portion 12 can be bonded along the length of the first tubular portion 13 or affixed at one or more points, such as junction 29.

FIGS. 6-7 depicts additional embodiments of the pusher assembly 30. that comprise a single continuous piece of tubing in which is modified to produce a more flexible second tubular portion 12 and a more kink-resistant first tubular portion 13. The embodiment of FIG. 6 depicts a single-piece tube in which the first tubular portion 13 is bumped down in diameter to form a thinner wall and therefore, more flexible first tubular portion 12. Extrusion techniques to vary the diameter of thermoplastic tubing are well know in the catheter arts. In the illustrative embodiment, an optional braid 23 is added to the second tubular portion 12 to allow it to be more flexible and less prone to kinking. An optional second member 15, such as that of FIG. 1, can be affixed over the transition zone 41 (or junction 29) between the two tubular portions 12,13 to facilitate negotiation of any kinks in the introducer catheter 11 that might form distal to that point. A thin layer 42 of polymer such as a shrink wrap or other type of polymer film, can be added to secure the braided portion 42 to the outer surface of the second tubular portion 12. In another embodiment, FIG. 7 depicts a pusher assembly 30 that has been extruded as two materials having different physical properties such as different degrees of column strength and/or flexibility. The first material, comprising the first tubular portion 13, blends with a second material comprising the second tubular portion 12 over a transition zone 41 from which the second tubular portion 12 extends distally, the second tubular portion 12 being generally more flexible than the proximal first tubular portion 13. The two materials must be compatible for co-extrusion and can include different polymers or two different compounds (e.g., different durometers) of the same polymer. Methods of co-extruding different polymers to form a single length of tubing are well known in the catheter arts.

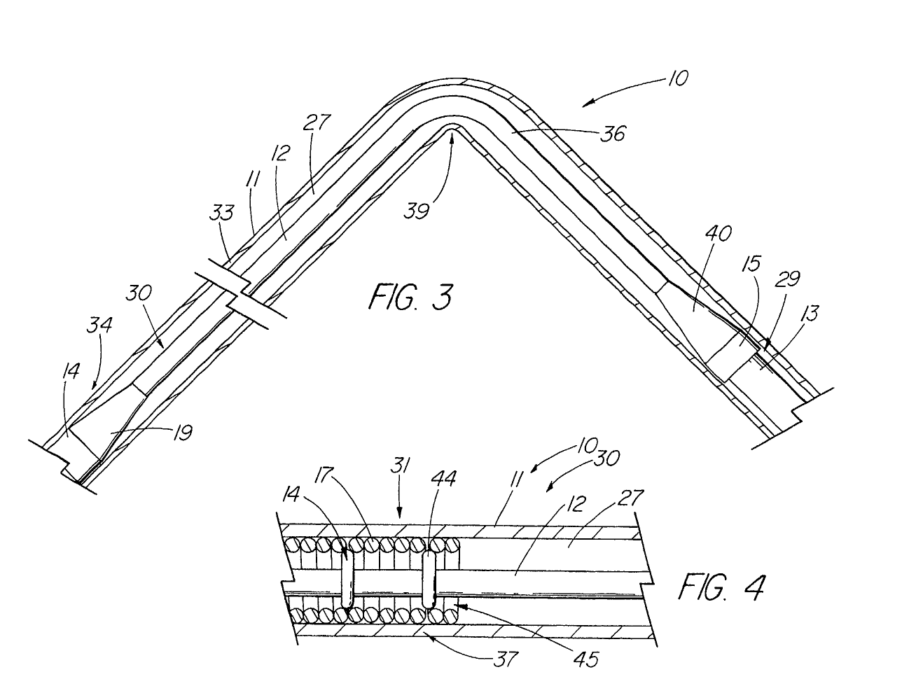

In assembling the illustrative stent introducer apparatus 10, the stent is loaded over the distal end 37 of the second tubular portion 12, and then distal tip 16 is placed thereover and bonded thereto, thereby holding the stent 17 in place. While the distal tip 16 is being affixed to the pusher assembly 30, pressure is applied such that the proximal end 31 of the stent 17 is forced tightly against the face 24 of the pusher member 14. This virtually eliminates any gap at the contact point 22, a gap which otherwise becomes a likely point of kinking when the introducer catheter is navigated through a severe bend, such as the common bile duct. The kink 39 generally occurs at that point along the introducer catheter 11 which experiences the greatest lateral bending forces during severe bending, this being largely determined by the degree of support provided by indwelling devices such as the pusher assembly 30 and the stent 17 itself. By reducing the weakness found at the contact 22 point between the pusher member 14 and the stent 17, the most likely location of any kink 39 (FIG. 3) in the introducer catheter 11 will be the flexible section 36 of the second tubular portion 12 which lies between junction 29 and the proximal end 31 of the stent 17. If a kink 39 develops within that section, it generally does not interfere with the ability of the pusher assembly 30 to slide within the introducer catheter 11 and expel the stent 17 therefrom. This is due to the pusher member 14 being distal to the kink 39 and in the case of the illustrative embodiment, the second tubular portion 12 is of a sufficiently small diameter such that the restriction of the introducer catheter lumen 27 still permits movement therethrough. Because this particular section of the introducer catheter 30 is flexible over an extended portion, any kink 39 that might occur is usually less severe than would be experienced in delivery systems of designs where the pusher system is stiff in comparison, and most of the bending force would be thus concentrated at the vulnerable contact point between the stent and the pusher member.

The stent introducer apparatus 10 of FIGS. 1-2 is designed to facilitate recapture, i.e., removal of the pusher assembly 30 back through the deployed stent. A number of points on a typical introducer apparatus have the potential of snagging and catching a strut, or otherwise becoming ensnared in the stent after delivery. To reduce the possibility of this occurring in the present invention, the proximal surface 18 includes a taper 18 that has been added to the distal tip 16 of the stent pusher assembly 30. In addition, proximal surface 19 of the pusher member 14 is also tapered as well. These tapers not only reduce the likelihood of an edge catching the stent during withdrawal, in the normal situation where the introducer catheter 11 is advanced by the physician after deployment to “recapture” the pusher assembly 30, but the tapers 18,19 also help guide the introducer catheter 11 over the distal tip 16 and pusher member 14 rather than having the distal end 21 of the introducer catheter 11 becoming temporarily caught up. In addition, the proximal tapers 16,18, especially that of the pusher member 14, help provide a guide to traverse any strictures during withdrawal of the pusher assembly 30 if the introducer catheter 11 becomes kinked. It should be understood that the invention includes other shapes or modifications of the proximal surfaces 18,19 of the distal tip and pusher member, other than a simple taper, that would produce a surface or edge that has a reduced likelihood or catching on the stent.

While the illustrative embodiment includes an expandable stent such as the SPIRAL Z™ Biliary Stent, knowledge of the type of stent to be used with the present invention, or how it is delivered is not essential for an understanding of the invention. Although the illustrative embodiment depicts a pusher member 14 to urge the stent 17 from the introducer catheter 11, alternative embodiments of the present invention could include a modified pusher assembly 30 that engages with the stent in another manner rather than pushing against the proximal end 31 of the stent 17. For example, the second tubular portion could extend into the lumen of the loaded stent and be frictionally engaged therewith. For example, FIG. 4 depicts a second embodiment of pusher member 14 that urges the stent 17 forward by engaging the struts or coils of the stent 17 from inside the stent lumen 45 via one or more engagement members 44 affixed over the shaft of the second tubular member 12. These engagement members can be made of plastic or metal and vary in shape, number, and distribution along the stent loading portion 35 of the second tubular portion 12. When the stent 17 is deployed and expands, the engagement members 44 no longer engage the stent 17, permitting withdrawal of the pusher member 30. Other embodiments could include a releasable engagement mechanism between the pusher assembly 30 and stent 17. Because of the variety of medical procedures for which this invention can be used, as well as the wide variety of stents that can be deployed, further modifications of the stent introducer apparatus of the present invention additional to the embodiments described herein are within the spirit of the invention and the scope of the claims. The invention contemplates embodiments comprising and consisting of the disclosed examples.

What is claimed is:

1. A stent introducer apparatus for use in target duct or vessels having an acute bend at a known general location in the body of a patient, comprising:
a pusher assembly that includes a pusher member configured to urge a preloaded stent from an introducer catheter into which it is slidably disposed, the pusher catheter assembly comprising a first and a second tubular portion, at least a portion of the second tubular portion extending distal of the first tubular portion, the second tubular portion including a flexible section, and a stent-carrying section located distal to the flexible section, the pusher member being located along the first tubular section at point that is either proximal to or within the stent-carrying section;
the flexible second tubular portion section having a preselected length and location along the pusher assembly such that when the apparatus and the preloaded stent are situated within an introducer sheath and are subjected to lateral bending stresses at the known general location, the flexible section of the second tubular portion traverses the known general location, whereby the likelihood of a kink occurring in the introducer catheter is greatest within a region corresponding to the region of greatest flexibility of the pusher assembly.

2. The stent introducer apparatus of claim 1, further including the stent preloaded within the distal portion of the introducer catheter, the stent further having a proximal end and a distal end.

3. The stent introducer apparatus of claim 2, wherein the pusher member includes a face having a diameter equal or greater than that of the stent while the stent is loaded in the introducer catheter, the proximal end of the stent and the face of the pusher member either closely adjacent to, or abutting one another.

4. The stent introducer apparatus of claim 2, wherein the second tubular portion further includes a stent loading section extending distally from the flexible section to at least the distal end of the stent.

5. The stent introducer apparatus of claim 2, wherein the stent is a self-expanding stent.

6. The stent introducer apparatus of claim 1 further including the introducer catheter.

7. The stent introducer apparatus of claim 1, wherein the second tubular portion has a smaller outer diameter than that of the first tubular portion.

8. The stent introducer apparatus of claim 7, wherein the second tubular portion comprises a metal-reinforced polymer material.

9. The stent introducer apparatus of claim 8 wherein the material comprises braided polyimide tubing.

10. The stent introducer apparatus of claim 9, wherein the second tubular portion comprises a nickel-titanium alloy.

11. The stent introducer apparatus of claim 9, wherein the second tubular portion includes a distal tip affixed about the distal end of the second tubular portion, and a pusher member affixed to an intermediate point along the second tubular portion that comprises a junction between the stent loading section and the flexible section, the stent loading section and the flexible section comprising a single continuous element, the stent being positioned over the stent loading section such the stent lies between, and is in contact with, both the distal tip and the pusher member.

12. An stent introducer apparatus, comprising:
an introducer catheter having a distal end and a distal portion;
a stent preloaded within the distal portion of the introducer catheter, the stent having a proximal end and a distal end;
a pusher assembly slidably disposed within the introducer catheter, the pusher assembly including both a second tubular portion having a first diameter and a first tubular portion having a second diameter and located proximate of the second tubular portion, the second diameter being greater than the first diameter;
a pusher member located along the second tubular portion, the pusher member including a face having a diameter equal to or greater than that of the stent preloaded in the introducer catheter;
a distal tip affixed about the distal end of the second tubular portion, the stent being tightly held between the distal tip and the face of the pusher member such that during deflection of the stent introducer apparatus, the point along the introducer catheter that receives the largest amount of bending stress and represents the more likely point where a kink would occur, is located proximal the pusher member.

13. The stent introducer apparatus of claim 12, wherein the second tubular portion comprises a metal-reinforced polymer material.

14. The stent introducer apparatus of claim 13 wherein the material comprises braided polyimide tubing.

15. The stent introducer apparatus of claim 12, wherein the second tubular portion comprises a nickel-titanium alloy.

16. The stent introducer apparatus of claim 12, wherein the second tubular portion further includes a stent loading section extending distal the flexible section, the stent loading section extending distally to at least the distal end of the stent.

17. The stent introducer apparatus of claim 16, wherein the stent is a self-expanding stent.

18. A stent introducer apparatus to be slidably disposed within an introducer catheter, wherein the stent introducer apparatus comprises a pusher member for exerting a force on the proximal end of a stent in order to expel the stent from the distal end of the catheter after the stent has been positioned within the distal end of the at least one of the introducer catheter or the stent introducer apparatus, wherein the stent introducer apparatus further comprises a pusher assembly to be mounted within the introducer catheter and to be controllable from the proximal region of the stent introducer apparatus in order to exert the force on the pusher member; characterized in that a distal section of the apparatus and catheter has an increased ability to laterally flex in comparison to the remaining section of the stent introducer apparatus and introducer catheter.

19. The stent introducer apparatus of claim 18, wherein the distal section extends either proximally from the pusher member to the remaining section, or extends from adjacent to the distal end of the apparatus and catheter to the remaining section.

20. The stent introducer apparatus of claim 19, wherein any tendency for the distal section to kink during the lateral flexing is compensated for by a second member mounted to the pusher assembly and shaped on its distal surface in such a manner as to enable the second member to open the kink to permit passage therethrough of the pusher assembly.

21. The stent introducer apparatus of claim 20, wherein any tendency of the distal section to kink or remain kinked during withdrawal of the pusher member and the pusher assembly is compensated for by shaping the proximal surface of the pusher member to open the kink and allow passage therethrough.

22. The stent introducer apparatus of claim 21, wherein the proximal surface of the second member is also shaped to enable the latter to open the kink and allow passage therethrough.

23. The stent introducer apparatus of claim 21, wherein the second member is fixed about the distal end of a first tube forming a part of the pusher assembly, the outer part of the first tube conforming to the inner diameter of the introducer catheter and thereby preventing kinking at any position proximal of the second member.

24. The stent introducer apparatus of claim 23, wherein the pusher assembly further comprises a second tube of significantly less outer diameter than that of the first tube, the second tube being of greater flexibility than the first tube and extending from at least the distal end of the first tube to at least the pusher member.

25. The stent introducer apparatus of claim 18, wherein the introducer catheter forms part of the apparatus, and includes sections of varying resiliencies.

26. A stent introducer apparatus, comprising:
an introducer catheter having a distal end and a distal portion;
a stent preloaded within the distal portion of the introducer catheter, the stent having a proximal end and a distal end;
a pusher assembly slidably disposed within the introducer catheter, the pusher assembly including both a second tubular portion comprising a metal braided reinforced polymer tube having a first outer diameter and including a distal end; and a first tubular portion having a second outer diameter and located proximal the second tubular portion, the second outer diameter being greater than the first outer diameter; the second tubular portion further comprising a stent-carrying section and a flexible section located proximal thereof, the stent-carrying section and flexible section divided by a pusher member that includes a face having a diameter equal to or greater than that of the stent preloaded in the introducer catheter; and
a distal tip affixed about the distal end of the second tubular portion, the stent being tightly held between the distal tip and the face of the pusher member such that during deflection of the stent introducer apparatus, the point along the introducer catheter that receives the largest amount of bending stress is located proximal the pusher member.
